SEQUENCE LISTING (9 sequences)

SEQ ID NO: 1 - DNA, 645 bp, Paenibacillus sp. 1794
gggtatctaatcctgtttgctccccacgctttcgcgcctcagcgtcagttacagcccagagagtcgccttcgccactggtgttcctccacatatctacgcatttcaccgctacacgtggaattccactctcctcttctgcactcaagtcacccagtttccagtgcgaaccaaggttgagccttggccttaaacaccagacttaaatgaccgcctgcgcgcgctttacgcccaataattccggacaacgcttgccccctacgtattaccgcggctgctggcacgtagttagccggggctttcttctcaggtaccgtcactccgatagcagttactctaccggacgttcttccctggcaacagagctttacgatccgaaaaccttcatcactcacgcggcgttgctccgtcaggctttcgcccattgcggaagattccctactgctgcctcccgtaggagtctgggccgtgtctcagtcccagtgtggccgttcaccctctcaggtcggctacgcatcgtcgccttggtgagccgttacctcaccaactagctaatgcgccgcaggcccatccgcaagtgacagattgctccgtctttcatcatcccctcaggagaggaaatgagatatccggtattagctcacgtt

SEQ ID NO: 2 - DNA, 374 bp, Paenibacillus sp. 1794
aacatcggtttgatcaactccttgtctacgttcgcacggatcaatgaatacggctttatcgaagctccgtaccgttgggtcgatccaaagacgggtgtcgtaacagatcagatcgactacctgacagctgatgaggaagacaactatgtggtcgcacaggcaaacgcaaagctgaatgaagacggcacgtttgcggaggatatggttatcgtccgttacaacaagcagtccgacaacatcttgccgatgccgagcgttcgcgtggattacatggacgtttcgccgaaacaggtcgtgtcagtcgcaacggcgctcattccgttcctcgagaacgatgactccgaccgcgccctgatgggtaccaacatgcaacg

SEQ ID NO: 3 - protein, 6 aa, Paenibacillus sp. 1794
KWNSWK

SEQ ID NO: 4 - protein, 6 aa, Paenibacillus sp. 1794
TTDYLM

SEQ ID NO: 5 - DNA, 18 bp, Artificial Sequence
  Oligonucleotide
aaatggaacagctggaaa

SEQ ID NO: 6 - DNA, 18 bp, Artificial Sequence
  Oligonucleotide
catcagatagtcggtcgt

SEQ ID NO: 7 - DNA, 1526 bp, Paenibacillus sp. 1794
  CDS (200)..(1432)
aaatatattttacaaataattggaaatcaggtagactaatagagaaactaatattaaagcgctttaactttcggagggggtgggactgcgttaatcctgtgatctgctgcccgcgccgatggcgtcccgggcggttacagctgagatgaaccgaggcataacctgtttcctaatctatgaaattaaaggagcgtaatgggtgatgaaaagaaataagagattttcattaagcggcaaagccgttctgatttgttgtctcgcttttctgatggttcccgccggatttgcgctggcggcgccgaacaagcctttccctcagcatacgacttacaccagcggctcgatcaagccgagtcatgtaacccagaccacgatggacgatgcagtcaaatccaaatgggacagctggaaatcagcttacctgaagccggccgggaccggcaaatattatgtgaagtacgattcaagcggcgcgacggtgtcagaagcgcatggctacggcatgctgctgaccgtgctgatggccggatacgacagcagcgcgcagacctattttaacggactgtataagtactacaccgagcatcccagcgacaataatccgtacttgatggcatggaaacagaacagcagctttcagaatgttgaaggagccgattccgccactgacggcgatatggacatcgcttattcgctcctgttggcggacaagcagtggggcagcagcggcacagtgaactacttgcaggcagccaaaaatatcatcaatgccattatgcaaaacgacgtgaaccagacgcaatggaccctccggcttggcgattgggctaccgacggttcctacgctacggcgacccgtccttcagacttcatgctgaaccatctgaaggcgttccagcaggcgaccggtgatacccggtggactaaggtcattgataaaacctactccattatcaattcgatttatacaaattacagtgcatcgacgggattgctgcccgactttgtcgtctactcaggaggcgcgtacaagccagcgccggccgacttcctggaagatacgaccgacggctcctacgcctataattcctgccgtacgccttggcggattacgaccgattacctgatgaccggcgacaaccgggccctgaatcagctgaaccagatgaacagctggatcaaaggcaaggtgaacagcaacccgggcgccgtcaaggacggatacaagttgaacggaagtgtcatcggttcgtataacagcggcgcctttttcgccccgttcggcgtcagcgccatgacttcctcgtcgaaccaaacctggctgaactccgtgtggacgtatacggccggaagctcaagcgaaggatactatgaagacagcatcaagctgttctcgctccttgtgatgtccggcaactggtggacgtattgatttctggggaagcctgtctatgggaaaataacatgaacagccccggccgcaattgggccggggctgtttggctatccccaagtgatgaagtggt

SEQ ID NO: 8 - protein, 410 aa, Paenibacillus sp. 1794
VMKRNKRFSLSGKAVLICCLAFLMVPAGFALAAPNKPFPQHTTYTSGSIKPSHVTQTTMDDAVKSKWDSWKSAYLKPAGTGKYYVKYDSSGATVSEAHGYGMLLTVLMAGYDSSAQTYFNGLYKYYTEHPSDNNPYLMAWKQNSSFQNVEGADSATDGDMDIAYSLLLADKQWGSSGTVNYLQAAKNIINAIMQNDVNQTQWTLRLGDWATDGSYATATRPSDFMLNHLKAFQQATGDTRWTKVIDKTYSIINSIYTNYSASTGLLPDFVVYSGGAYKPAPADFLEDTTDGSYAYNSCRTPWRITTDYLMTGDNRALNQLNQMNSWIKGKVNSNPGAVKDGYKLNGSVIGSYNSGAFFAPFGVSAMTSSSNQTWLNSVWTYTAGSSSEGYYEDSIKLFSLLVMSGNWWTY

SEQ ID NO: 9 - protein, 378 aa, Paenibacillus sp. 1794
APNKPFPQHTTYTSGSIKPSHVTQTTMDDAVKSKWDSWKSAYLKPAGTGKYYVKYDSSGATVSEAHGYGMLLTVLMAGYDSSAQTYFNGLYKYYTEHPSDNNPYLMAWKQNSSFQNVEGADSATDGDMDIAYSLLLADKQWGSSGTVNYLQAAKNIINAIMQNDVNQTQWTLRLGDWATDGSYATATRPSDFMLNHLKAFQQATGDTRWTKVIDKTYSIINSIYTNYSASTGLLPDFVVYSGGAYKPAPADFLEDTTDGSYAYNSCRTPWRITTDYLMTGDNRALNQLNQMNSWIKGKVNSNPGAVKDGYKLNGSVIGSYNSGAFFAPFGVSAMTSSSNQTWLNSVWTYTAGSSSEGYYEDSIKLFSLLVMSGNWWTY

(19) United States
(12) Patent Application Publication
(10) Pub. No.: US 2013/0337541 A1
(43) Pub. Date: Dec. 19, 2013

(54) THERMOSTABLE CHITOSANASE

(71) Applicants: Ryszard Brzezinski, Sherbrooke (CA); Mélanie Fortin, Sherbrooke (CA); Mina Zitouni, Sherbrooke (CA)

(72) Inventors: Ryszard Brzezinski, Sherbrooke (CA); Mélanie Fortin, Sherbrooke (CA); Mina Zitouni, Sherbrooke (CA)

(73) Assignee: SOCPRA Sciences et Genie S.E.C., Sherbrooke, QC (CA)

(21) Appl. No.: 13/979,500

(22) PCT Filed: Jan. 13, 2012
(86) PCT No.: PCT/CA2012/050022

§ 371 Date: Aug. 30, 2013

Related U.S. Application Data
(60) Provisional application No. 61/437,204, filed on Jan. 28, 2011.

(51) Int. Cl. C12N 9/24

(52) U.S. Cl. CPC C12N 9/2402
USPC 435/200; 536/23.2; 435/320.1; 435/252.3; 435/274; 435/252.33

(57) ABSTRACT

The present application concerns a thermostable chitosanase, originally identified in Paenibacillus sp., having an optimal temperature of about 80° C. when measured at a reaction time of about 10 minutes. Also contemplated are variants of this chitosanase (sharing more than 81% identity) as well as fragments thereof. The present application further concerns nucleic acid molecules encoding the chitosanase, vector comprising them as well as host cell expressing them. Methods of producing the thermostable chitosanase as well as using it for generating low molecular weight chitosan and chitosan oligosaccharides are also disclosed.

### CROSS-REFERENCE TO RELATED APPLICATIONS AND DOCUMENTS This is application claims priority from U.S. provisional patent application 61/437,204, filed on Jan. 28, 2011 and herein incorporated in its entirety.

This application contains a sequence listing submitted electronically. The content the electronic submission is incorporated by reference in this application in its entirety.

This application refers to a biological deposit made on Jan. 11, 2011 under the Budapest Treaty which was attributed Accession Number 110111-01 from the International Authority of Canada. The content of this biological deposition is incorporated by reference in this application in its entirety.

### FIELD OF THE INVENTION

This application relates to a novel thermostable chitosanase as well as to its associated nucleic acid molecules and its uses for reducing the molecular weight of chitosan.

### BACKGROUND

Chitosan is a polysaccharide obtained by N-deacetylation of chitin. In industrial scale procedures, chitosan is obtained from chitin by alkali treatment of crustacean shells. Chitosan is also present in nature in the cell walls of some fungi and algae and in insects. However, these natural sources have not been used, so far, in industrial applications. Chitosan is mainly composed of β-1,4-linked D-glucosamine units with a variable content of N-acetyl-D-glucosamine units. The percentage of N-acetyl-D-glucosamine units is defined as the degree of N-acetylation of chitosan (“DA”), while the percentage of D-glucosamine units is also called the degree of deacetylation (“DDA”) of chitosan. Most commercial preparations of chitosan are characterized by dda values between 70 and 99%.

Chitosan is unique among polysaccharides because it carries amino groups which are positively charged in mildly acidic aqueous solution (pH <6.2). Most biological properties of chitosan result from the presence of these positively charged groups. The amino groups can also be coupled to various chemical groups, resulting in a large family of chitosan derivatives described by various authors. Chemical and physical properties of chitosan, as well as of its parent polymer chitin, are largely described in the literature.

Various forms of chitosan are under intense study due to their many potential applications in agriculture, biotechnology, in fundamental research or as health products. Two parameters of chitosan molecules were shown to strongly influence its usefulness/properties for a given application: the degree of deacetylation and the molecular weight (also referred to as the “degree of polymerization”). Other parameters that may be important for determining the chitosan's characteristics include the distribution of the molecular weight (“polydispersity”) and the pattern of N-acetylation.

The degree of deacetylation can be controlled, to some extent, by the conditions (time, temperature) of the alkaline treatment of chitin. Enzymatic methods of chitin treatment by chitin deacetylases are under development. Fully deacetylated chitosan can be obtained by chemical treatment of chitin; however the toxic reagents used in this procedure will probably limit its application to basic laboratory research.

Native chitosan molecules, as isolated from natural organisms or obtained after alkaline N-deacetylation of chitin, are of high molecular weight (in the range of millions of daltons) with degrees of polymerisationpolymerization reaching several thousand units. While various applications were described for such high molecular weight chitosans (HMWC), for most applications a narrower, optimal range of molecular weight is under consideration. Chitosan polymers with shorter than native chains are often divided into low molecular weight chitosans (LMWC), with a range of molecular weight roughly between 5 kDa and 100 kDa and chitooligosaccharides (or chitosan oligosaccharides; CHOS) with a lower limit of 0.4 kDa (glucosamine dimer) while the higher limit is less defined (5-10 kDa). The CHOS are fully soluble in water and are essentially prepared as undefined mixtures of oligomeric molecules of various molecular weights and degrees of N-acetylation. However, methods of characterization and purification of defined CHOS are in rapid development. Both LMWC and CHOS have numerous potentials applications in medicine, agriculture, environment, besides their use as reagents in basic and applied research. Well documented applications include their use in protection against plant disease, their use as a hypocholesterolemic agent, as an anti-inflammatory agent, as an antimicrobial and antifungal product. Other applications include tumor growth inhibition, vector of genetic material for cell transformation as well as an accelerator of wound healing.

The molecular weight of chitosan can be decreased by various methods. Several chemical and physical methods, such as hydrolysis with acids or ultrasonic degradation were proposed. However such methods offer little control on the molecular weight of the final product. Furthermore, chemical and physical methods often modify the degree of deacetylation of chitosan during the process. For these reasons, enzymatic methods for decreasing the molecular weight of chitosan show would be beneficial. Also, enzymatic methods are less expensive in energy and produce biodegradable waste residues, they thus can be considered as “green technologies”. Enzymes able to hydrolyze glycosidic links in chitosan include chitosanases, chitinases, lysozymes and a few other, less specific enzymes.

Native chitosan is a polymer of high molecular weight and yields highly viscous solutions even when dissolved at relatively low concentrations. This problem is largely known in the biotechnology of other polymeric polysaccharides, such as starch or beta-glucans. Viscosity of the polymeric solutions can be decreased simply by raising the temperature during the enzymatic process. Known chitosanases have an optimal enzymatic temperature between 50 and 60° C. In industrial settings, the usual temperature used for performing this enzymatic reaction is 50° C. Some available chitosanases have been shown to be active at higher temperatures, but the use of these chitosanases at temperatures higher than 70° C. have not shown to be possible.

There is a need in an industrial setting, to enzymatically cleave chitosan at a temperature of 70° C. or more with a thermostable chitosanase. Native chitosan is highly viscous in aqueous solution and its dissolution is enhanced by increasing temperature. By performing the enzymatic reaction at 70° C., more chitosan can be dissolved and there is approximated a 25% gain in efficiency. Thermostable chitosanase enzymes are needed to proceed with hydrolysis at such high temperatures. This would allow dissolving chitosan at higher concentrations, increasing the treatment capacity of bioreactors. Furthermore, enzymes increase their velocity at high temperatures (as long as they are stable during the time of the process) which results in enzyme savings, as the quantity of the enzyme needed to hydrolyze a given amount of polymer can be lowered. Finally, application of higher temperatures during enzymatic hydrolysis will substantially decrease the possibility of bacterial contamination of the hydrolysis product.

### BRIEF SUMMARY

The present applicable provides a chitosanase having an enzymatic activity that is stable at elevated temperatures. This chitosanase can be advantageously used in processes including enzymatic cleavage steps at elevated temperatures to produce, for example, low molecular weight chitosan or chitosan oligosaccharides.

In a first aspect, the present application provides an isolated thermostable chitosanase comprising an amino acid sequence having more than 81% identity to the amino acid sequence of SEQ ID NO: 9. The chitosanase has preferably an optimal temperature of about 80° C. when measured at a reaction time of about 10 minutes. In an embodiment, the thermostable chitosanase consists essentially of an amino acid sequence having more than 81% identity to the amino acid sequence of SEQ ID NO: 9. In yet another embodiment, the thermostable chitosanase consists of an amino acid sequence having more than 81% identity to the amino acid sequence of SEQ ID NO: 9. In a further embodiment, the thermostable chitosanase comprises the amino acid sequence of SEQ ID NO: 9. In still a further embodiment, the thermostable chitosanase consists essentially of the amino acid sequence of SEQ ID NO: 9. In yet a further embodiment, the thermostable chitosanase consists of the amino acid sequence of SEQ ID NO: 9. In an embodiment, the isolated thermostable chitosanase further comprises (or contains) at least one of a glutamate residue at position 64 or a glutamate residue at position 254. In another embodiment, the isolated thermostable chitosanase further comprises (or contains) a glutamate residue at position 64 and a glutamate residue at position 254. In yet another embodiment, the isolated thermostable chitosanase further comprises (or contains) at least one of (or at least two of, or at least three of, or at least four of, or at least five of, or at least six of) an aspartate residue at position 56, a tryptophan residue at position 108, an aspartate residue at position 121, an aspartate residue at position 125, a tryptophan residue at position 177, a tyrosine residue at position 263 and/or a tyrosine residue at position 359. In another embodiment, the isolated thermostable chitosanase further comprises (or contains) an aspartate residue at position 56, a tryptophan residue at position 108, an aspartate residue at position 121, an aspartate residue at position 125, a tryptophan residue at position 177, a tyrosine residue at position 263 and a tyrosine residue at position 359. In an embodiment, the isolated thermostable chitosanase is derived from Paenibacillus sp. 1794 deposited at the International Depository Authority of Canada (IDAC) on January 11.2O11 and having IDAC Accession Number 110111-01.

In a second aspect, the present application provides an isolated thermostable chitosanase comprising an amino acid sequence having more than 81% identity to the amino acid sequence of SEQ ID NO: 9, wherein said chitosanase has an optimal temperature of about 80° C. when measured at a reaction time of about 10 minutes, wherein the amino acid sequence of the isolated thermostable chitosanase comprises a glutamate residue at position 64, a glutamate residue at position 254, an aspartate residue at position 56, a tryptophan residue at position 108, an aspartate residue at position 121, an aspartate residue at position 125, a tryptophan residue at position 177, a tyrosine residue at position 263 and/or a tyrosine residue at position 359. In an embodiment, the isolated thermostable chitosanase is derived from Paenibacillus sp. 1794 deposited at the International Depository Authority of Canada (IDAC) on January 11, 2011 and having IDAC Accession Number 110111-01.

In a third aspect, the present application provides an isolated nucleic acid molecule encoding the isolated chitosanase described herein. In an embodiment, the isolated nucleic comprises nucleotides 200 to 1432 of the nucleotide sequence of SEQ ID NO: 7. In another embodiment, the isolated nucleic acid consists essentially of nucleotides 200 to 1432 of the nucleotide sequence of SEQ ID NO: 7. In yet another embodiment, the isolated nucleic acid consists of nucleotides 200 to 1432 of the nucleotide sequence of SEQ ID NO: 7.

In a fourth aspect, the present application provides a vector encoding the isolated chitosanase described herein and/or the isolated nucleic acid of encoding the isolated chitosanase.

In a fifth aspect, the present application provides a host cell expressing a recombinant form of the isolated thermostable chitosanase described herein. In an embodiment, the host cell comprises the isolated nucleic acid described herein or the vector described herein. In yet another embodiment, the host cell is a bacterial host cell, such as, for example, Escherichia coli.

In a seventh aspect, the present application provides a method of producing a thermostable chitosanase. Broadly, the method comprises placing the host cell described herein in a culture medium under conditions sufficient to allow the production of the thermostable chitosanase. In an embodiment, the method further comprises recuperating the thermostable chitosane from the culture medium.

In an eighth aspect, the present application provides a method of reducing the molecular weight of a chitosan molecule. Broadly, the method comprises contacting the isolated chitosanase described herein or produced by the method described herein with the chitosan molecule under conditions sufficient to allow the cleavage of the chitosan molecule by the isolated chitosanase. In an embodiment, the contacting occurs at a temperature of between about 60° C. to 80° C., 70° C. to 75° C., preferably about 70° C.

In a ninth aspect, the present application provides a method of producing a low-molecular weight chitosan. Broadly, the method comprises contacting the isolated chitosanase described herein or produced by the method described herein with a chitosan molecule under conditions sufficient to allow the cleavage of the chitosan molecule by the isolated chitosanase into the low molecular weight chitosan. In an embodiment, the contacting occurs at a temperature of between about 60° C. to 80° C., 70° C. to 75° C., preferably about 70° C. In yet another embodiment, the method further comprises purifying the low molecular weight chitosan.

In a tenth aspect, the present application provides a method of producing a chitosan oligosaccharide, said method comprising contacting the isolated chitosanase described herein or produced by the method provided herein with a chitosan molecule under conditions sufficient to allow the cleavage of said chitosan molecule by said isolated chitosanase into said chitosan oligosaccharide. In an embodiment, the contacting occurs at a temperature of between about 60° C. to 80° C., 70° C. to 75° C., preferably about 70° C. In yet another embodiment, the method further comprises purifying the chitosan oligosaccharide.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 presents the nucleotide and amino acid sequences presented in the application. (A) Partial sequence of the Paenibacillus sp. 1794 gene encoding the ribosomal 16S RNA (645 bp). Sequence is also shown as SEQ ID NO: 1. (B) Partial sequence of the Paenibacillus sp. 1794 gene encoding the beta-subunit of the RNA polymerase gene (rpoB) (374 bp). Sequence is also shown as SEQ ID NO: 2. (C) DNA sequence encoding the chitosanase Csn1794 together with upstream and downstream intergenic segments. The csn1794 coding segment begins with the underlined start codon GTG (nucleotides 200-202) and ends with the underlined stop codon TGA (nucleotides 1430-1432). Sequence is also shown as SEQ ID NO: 7. (D) DNA sequence encoding the chitosanase Csn1794 together with its translation into amino acids. DNA sequence is also shown as SEQ ID NO: 7 and amino acid sequence is shown as SEQ ID NO: 8. (E) Amino acid sequence of the chitosanase Csn1794. The predicted and underlined signal peptide (32 residues-long) cleaved out during the process of secretion is underlined. Complete amino acid sequence (with signal peptide) is shown in SEQ ID NO: 8 and mature amino acid sequence (without signal peptide) is shown in SEQ ID NO: 9.

FIG. 2 shows the relative activity of chitosanase Csn1794 against chitosans of various degrees of deacetylation (DDA). Activity against the chitosan from Sigma (having a DDA=0.84) is taken as a reference (relative activity=1).

FIG. 3 shows the relative activity of chitosanase Csn1794 at various pH. Activity at pH 4.6 is taken as a reference (100%).

FIG. 4 illustrates the activity of chitosanase Csn1794 as a function of temperature. The activity is expressed as a concentration of reducing sugars (μmol/mL) generated for various reaction times in enzymatic reactions carried out by Csn1794. Reaction time: ▴: 10 min; ▪: 5 min; ♦; 3 min.

FIG. 5 shows the velocity (V) of enzymatic reaction catalyzed by Csn1794 (μmol/10 min) as a function of substrate concentration (mg/mL).

FIG. 6 illustrates a time-course of hydrolysis product accumulation at high temperatures. Concentration of reducing sugars (μmol/mL) is shown in function of time (min). Product concentration was determined by the reducing sugar assay. Hydrolysis was carried out at the following temperatures: ▪: 70° C.; : 75° C.; ♦: 78° C.

### DETAILED DESCRIPTION

In accordance with the present invention, there is provided a thermostable chitosanase, a nucleic acid molecule encoding same, a vector comprising the nucleic acid molecule, a host cell capable of producing same as well as associated method for the production of the enzyme and its use in the cleavage of chitosan. The thermostable chitosanase has an optimal temperature of 80° C., when measured during a 10-minute reaction.

In order to select a novel thermostable chitosanase, commercial compost was enriched with a mixture of chitosan powder and salts and incubated for a couple of weeks. The incubation was conducted at three different temperatures. A screening of more than a 2 000 microbial colonies was performed. Only those capable of hydrolyzing chitosan were selected. The chitosanases obtained were isolated and their thermostability was determined. The bacterial isolate (herein referred to as Paenibacillus sp. 1794) having the most thermostable chitosanase was selected for further characterization. Under certain specific conditions, this isolate can produce up to 12 enzymatic units per mL of culture. The amino acid entity of the thermostable chitosanase derived from Paenibacillus sp. 1794 as well as its corresponding nucleic acid was determined.

### Thermostable Chitosanase

The present application provides a thermostable chitosanase. This thermostable chitosanase's amino acid sequence has more than 81% identity to the amino acid of SEQ ID NO: 9 and has an optimal temperature of about 80° C., when measured during a 10-min reaction.

The thermostable chitosanase presented herewith has more than 81% identity/homology to the amino acid sequence of SEQ ID NO: 9. As used herein, the term “more than 81% identity” specifically excludes amino acid sequences having exactly 81% or less identity to the amino acid sequence of SEQ ID NO: 9. In an embodiment, the amino acid sequence of the thermostable chitosanase has more than 81%, 82%, 83%, 84%, 85%, 86%, 87%, 88%, 89%, 90%, 91%, 92%, 93%, 94%, 95%, 96%, 97%, 98% or 99% identity to the amino acid sequence of SEQ ID NO: 9. In another embodiment, the thermostable chitosanase has more than 81% but less than 82%, 83%, 84%, 85%, 86%, 87%, 88%, 89%, 90%, 91%, 92%, 93%, 94%, 95%, 96%, 97%, 98% or 99% identity to the amino acid sequence of SEQ ID NO: 9. In another embodiment, the thermostable chitosanase's amino acid sequence comprises the amino acid sequence of SEQ ID NO: 9. In yet another embodiment, the amino acid sequence of the thermostable chitosanase consists of SEQ ID NO: 9.

“Identity”, as known in the art, is a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed). 1988, Computational Molecular Biology, Oxford University Press, N.Y.; D. W. Smith (ed), 1993, Biocomputing. Informatics and Genome Projects, Academic Press, N.Y.; A. M. Griffin and H. G. Griffin, H. G (eds), 1994, ComputerAnalysis of Sequence Data, G. von Heinje, 1987, Sequence Analysis in Molecular Biology, Academic Press; M. Gribskov and J. Devereux (eds), 1991, Sequence Analysis Primer, M Stockton Press, N.Y.; Carillo and Lipman (1988, SIAM J. Applied Math., 48:1073).

The percentage of identity is determined over a specific portion of the amino acid sequence SEQ ID NO: 9, usually the entire length of the amino acid sequence of SEQ ID NO: 9. In order to determine the percentage of identity between any amino acid sequence or the amino acid sequence of SEQ ID NO: 9, various tools are known to those skilled in the art. For example, one can use the Protein Blast with the blastp algorithm, a software which is freely accessible through the NCBI's web site (http://blast.ncbi.nlm.nih.gov/Blast.cgi?PROGRAM=blastp&BLAST_PROGRAMS=blast p&PAGE_TYPE=BlastSearch&SHOW_DEFAULTS=on&LINK_LOC=blasthome).

In order to determine the optimal temperature of a chitosanase, various methods are known in the art. For example, one can choose to incubate the enzyme in the presence of its substrate at a specific temperature and determine the amount of cleavage product in function of a specified time period. The results obtained are then used to estimate the optimal temperature. In an embodiment, the optimal temperature for the enzymatic activity of Csn1794 can be 80° C. for a 10 min-enzymatic reaction and/or 85° C. for a 5 min- or 3 min-enzymatic reaction.

The thermostable chitosanases described herein do possess a chitosanase activity at a high temperature, e.g. they are able to cleave the chitosan within its chain. More specifically, the thermostable chitosanase has an endo-chitosanase activity. The thermostable chitosanases described herein can possess other enzymatic activity, such as the cleavage of other types of bounds in chitosan or in another polymer (cellulase activity for example). However, the thermostable chitosanase has a higher chitosanase activity than any other enzymatic activity (such as cellulase activity).

The thermostable chitosanases described herein are provided in a soluble form, e.g. they are not covalently bound to another entity or to a surface. The thermostable chitosanases are designed to be added to a reaction mixture in a soluble form. The enzymatic activity of the thermostable chitosanase described herein is measured in a soluble form (e.g. not covalently bound to a substrate). However, in order to increase their enzymatic activity, it is possible to provide a thermostable chitosanase bound to a solid support.

The thermostable chitosanases described herein are particularly advantageous because they remain thermostable even in the presence of their hydrolysis substrate and/or have a relatively long half-life. For example, in conditions close to those used in an industrial setting (chitosan concentration >5 g/L dissolved in 0.4 M acetic acid; pH 4.4-4.8) the thermostable chitosanase having the amino acid sequence of SEQ ID NO: 9 retained close to 100% of its enzymatic activity during at least six (6) hours at 70° C. At 78° C., the half-life (T_{1/2}) of the thermostable chitosanase having the amino acid sequence of SEQ ID NO: 9 is about 192 minutes.

In an embodiment, the isolated chitosanase can also comprise a signal peptide to facilitate its secretion from an host cell that is being cultured under appropriate conditions. For example, the signal peptide set forth between residues 1 and 32 of SEQ ID NO: 8 can be operatively linked to the isolated thermostable chitosanase described herein.

Amino acid variants of the thermostable chitosanase set forth in SEQ ID NO: 9 can also be used. Those amino acid variants should retain a endo-chitosanase activity and should have an optimal temperature of 80° C. when determined in a 10 min reaction. In an embodiment, amino acid variants also show at least one of the following biochemical characteristics associated with the native sequence: member of the GH8 family, molecular weight between 38 and 44 kDa, specific activity between 120 and 125 U/mg of protein when measured at 37° C., optimal pH 5.0, close-to-optimal pH between 3.8 and 6.5, kₘ=0.03 mg/ml chitosan (DDA 84%), K_{cat}=7028/min, T_{1/2} at 78° C. between 190 and 195 minutes.

Conservative substitutions can be introduced into the sequence of SEQ ID NO: 9, but other substitutions are also contemplated. It is of interest that certain amino acid residues in the sequence of SEQ ID NO: 9 preferably should not be modified/substituted because they are either involved in the catalytic site and/or in the substrate binding site and seem to be of importance for enzymatic activity.

The thermostable chitosanase isolated from Paenibacillus sp. 1794 is from the GH8 family. One member of this family, isolated from Bacillus sp. K-17, was crystallized and the amino acids involved in the active site as well as the substrate recognition site were identified. Briefly, for the chitosanase isolated from Bacillus sp. K-17, the amino acid residues implicated in the catalytic site were identified as Glu122 and Glu309, whereas the amino acid residues involved in the substrate binding were identified as Glu107, Trp166, Asp179, Asp183, Trp235, Glu309, Tyr318 and Phe413. When comparing the polypeptide sequence of the chitosanase from Bacillus sp. K-17 and the thermostable chitosanase of Paenibacillus sp. 1794, it was deduced, in the polypeptide still comprising the signal peptide (SEQ ID NO: 8), that amino acid residues Glu96 and Glu286 are involved in the catalytic site, whereas amino acid residues Asp88; Trp140; Asp153; Asp157; Trp209; Tyr295 and Tyr391 are involved in the substrate binding site.

Therefore, the present application also provides an isolated thermostable chitosanase variant which comprises one or both amino acid residues involved in the catalytic site (e.g. in SEQ ID NO: 9, a glutamate residue corresponding to position 64 and/or a glutamate residue corresponding to position 254). There is also provided an isolated thermostable chitosanase variant which comprises one or a combination of the amino acid residues involved in the substrate binding site (e.g. in SEQ ID NO: 9, an aspartate residue corresponding to position 56, a tryptophan residue corresponding to position 108, an aspartate residue corresponding to position 121, an aspartate residue corresponding to position 125, a tryptophan residue corresponding to position 177, a tyrosine residue corresponding to position 263 and/or a tyrosine residue corresponding to position 359).

In yet another embodiment, fragments of the thermostable chitosanase set forth in SEQ ID NO: 9 are considered. As used herein, the term “fragment” refers to a sequence that is shorter than the one set forth in SEQ ID NO: 9. In an embodiment, this fragment may share more than 81% identity to a fragment of SEQ ID NO: 9 having substantially the same length as the fragment. In still another embodiment, the fragment can be identical to a fragment of the sequence of SEQ ID NO: 9. Those fragments should retain an endo-chitosanase activity and should have an optimal temperature of 80° C. for a 10-min reaction. In an embodiment, these fragments can be associated (in a covalent or non-covalent manner) to another entity, such as, for example, another polypeptide to form a chimeric protein. In an embodiment, the fragments or the chimeric proteins containing such fragments also show at least one of the following biochemical characteristics associated with the native sequence: catalytic module is a member of the GH8 family, molecular weight lower than 41 kDa, optimal pH between 4.2 and 4.8, The optimal pH for this enzyme is 4.7-4.8, but it still retains a close-to-maximal activity at a pH between 3.8 and 6.5.

Fragments of the isolated thermostable chitosanase variant can comprise one or both amino acid residues involved in the catalytic site of the native enzyme (e.g. glutamate residue at position 64 and/or a glutamate residue at position 254). Alternatively or optionally, the fragments can also comprise one or a combination of the amino acid residues involved in the substrate binding site (e.g. an aspartate residue at position 56, a tryptophan residue at position 108, an aspartate residue at position 121, an aspartate residue at position 125, a tryptophan residue at position 177, a tyrosine residue at position 263 and/or a tyrosine residue at position 359).

In an embodiment, the thermostable chitosanase can be derived from Paenibacillus sp. 1794, which was deposited at the International Depository Authority of Canada (IDAC) on January 11, 2011 and having IDAC Accession Number 110111-01.

In an embodiment, the isolated thermostable chitosanase is Csn1794. This chitosanase is a protein secreted in the extracellular medium. In its secreted form, it has an estimated molecular weight of 41.8 kilodaltons. The enzyme has an endo-chitosanase activity, e.g. it is able to cleave the chitosan's glycosidic beta-1,4 bonds within the chitosan chain. The isolated chitosanase has a specific activity of about 122.4 U/mg of protein when measured at 37° C. One (1) unit of enzyme is defined as the quantity of enzyme capable of catalyzing the liberation of 1 μmol of reducing sugars in one minute at 37° C. The optimal temperature for the enzymatic activity of Csn1794 is 80° C. for a 10 min-enzymatic reaction and 85° C. for an 5 min-enzymatic reaction. The optimal pH for this enzyme is 4.7-4.8, but it still retains a close-to-maximal activity at a pH between 3.8 and 6.5. Kinetic parameters were determined as follows: kₘ=0.03 mg/ml chitosan (DDA 84%), K_{cat}=7028/min. Kᵢ=1.66 mg/mL. The Csn1794 chitosanase can enzymatically cleave chitosane having different DA (between 1% and 35%) while retaining between 80 et 100% of its maximal activity.

In another embodiment, the thermostable chitosanase can have the amino acid sequence as set forth in SEQ ID NO: 8 (with signal peptide) or SEQ ID NO:9 (without the signal peptide. The amino acid sequence of the thermostable chitosanase can also consist in the amino acid sequence set forth in SEQ ID NO: 8 or SEQ ID NO: 9.

In still another embodiment, the amino acid sequence of the thermostable chitosanase can be the amino acid sequence of Csn1794. The Csn1794 polypeptide corresponds to a 410 amino acid-long sequence (set forth in SEQ ID NO: 8), as deduced from the csn1794 gene. The Csn1794 sequence is shortened to 378 amino acids when the protein is in its extracellular form when its signal peptide is cleaved during the secretion process. Because of its amino acid sequence, this protein is classified in the glycoside hydrolase 8 family (also known as the GH8 family).

### Thermostable Chitosanase-Encoding Nucleic Acids and Vectors

The present application provides isolated nucleic acids encoding the thermostable chitosanase as well as vectors comprising the nucleic acid. Optionally, the sequence of the nucleic acid can be optimized for codon usage and recognition depending on the host cell that is considered for expression of the thermostable chitosanase gene and protein.

Vectors (also referred to as expression vectors) can be derived from retroviruses, adenovirus, herpes or vaccinia viruses or from various bacterial or fungal plasmids and cosmids which may be used for the expression of the thermostable chitosanase. Methods which are well known to those skilled in the art can be used to construct recombinant vectors which will express the isolated nucleic acid sequence and ultimately lead to the production of the thermostable chitosanase. More specifically, the vector can comprise a promoter sequence, preferably located upstream of the nucleic acid encoding the thermostable chitosanase. The promoter sequence can be the native promoter of the csn1794 gene (or a portion thereof). The promoter sequence can be another promoter that would be recognized by the host cell carrying the vector. The promoter can be inducible or constitutive. In an embodiment, the vector can also comprise a selection marker to facilitate the identification of host cells carrying the vector. Optionally, the vector can further comprise a fusion peptide or protein, operatively linked to the coding-sequence of the thermostable chitosanase. The use of the fusion peptide or protein may be advantageous for the isolation of the thermostable chitosanase.

In an embodiment, the isolated nucleotide acid comprises the gene encoding the thermostable chitosanase Csn1794, herein referred to as csn1794. The nucleic acid sequence of the csn1794 gene is 1233 nucleotide-long and is presented between residues 200 and 1432 of SEQ ID NO: 7.

### Thermostable Chitosanase-Producing Host Cell

The present application also provides a host cell expressing the isolated thermostable chitosanase and/or comprising a vector encoding the isolated thermostable chitosanase. This host cell can serve for the production (e.g. mass production) of the thermostable chitosanase. The host cell is capable of expressing the thermostable chitosanase having a chitosanase activity at a high temperature. In an embodiment, the host cell is also capable of secreting such thermostable chitosanase in the growth medium to facilitate the recuperation and, optionally, further purification of the thermostable chitosanase.

In an embodiment, this host cell can be, for example, a bacterium preferably capable of expressing and secreting the isolated thermostable chitosanase. This bacterium can be, for example, from the genus Escherichia (such as Escherichia coli) or Paenibacillus (such as Paenibacillus sp. 1794) or Bacillus (such as Bacillus subtilis). In another embodiment, the host cell can be an actinobacterium such as Amycolatopsis or Streptomyces. In another embodiment, the host cell can be a yeast cell, such as, for example. Saccharomyces cerevisiae, Pichia pastoris or Kluyveromyces lactis. In yet another embodiment, the host cell can be a eukaryotic cell, such as, for example, a plant or a mammalian cell line.

A particular Paenibacillus, Paenibacillus sp. 1794, was identified and is characterized herein and could be used for the production (e.g. mass production) of the thermostable chitosanase. This isolate was deposited at the International Depositary Authority of Canada (IDAC) on January 11, 2011 and was attributed Accession Number 110111-01. However, other bacterial isolates having the identifying characteristics (listed below) of Paenibacillus sp. 1794 could also be used.

Morphological and biochemical characterisitics of Paenibacillus sp. 1794. This rod-shape bacterium is catalase positive and oxydase positive. Its optimal growth temperature is 45° C., whereas its maximal growth temperature is 55° C. It can grow, albeit slowly, in the presence of 5% NaCl. This bacterium is capable of metabolizing mannitol and sorbitol as energy sources. It cannot metabolize methyl red and cannot produce gas from the utilization of glucose. No hemolysis was detected. It can produce acids from D-glucosamine, N-acetyl-D-glucosamine, L-arabinose, D-ribose, D-xylose and D-glucose, but not from glycerol and sorbitol. This isolate specifically differs from Paenibacillus cineris by its reduced ability to grow in the presence of 5% NaCl, its ability to utilize D-glucosamine and its lack of ability to utilize sorbitol to produce acids.

Molecular taxonomy. By sequencing the 16S ribosomal RNA of Paenibacillus sp. 1794, it was realized that this isolate belonged to the genus Paenibacillus, but that its sequence differed by a single nucleotide from the Paenibacillus cineris species. By sequencing a fragment of the rpoB gene coding for the subunit beta of the RNA polymerase (also known as the DDGE fragment), it was determined that this isolate differed at various nucleotides from other known isolate, including Paenibacillus cineris. It was concluded that this was a completely novel and uncharacterized isolate.

### Method of Producing Low Molecular Weight Chitosan and Chitosan Oligosaccharide

The present application also provides methods of cleaving chitosan molecules to generate either low molecular weight chitosan (usually between 5 and 100 kDa) or chitosan oligosaccharides (usually between 0.4 and 5-10 kDa). The methods presented herein use the thermostable chitosanase described herein for reducing the molecular weight of the chitosan. The method presented herein should be conducted under conditions allowing enzymatic activity of the thermostable chitosanase. Such condition can include, but are not limited to, temperature, pH, reaction medium, presence of substrate, absence of inhibitors, etc. The method can also optionally comprise a step for the recuperation and purification of the products of the enzymatic reaction (e.g. LMWC or CHOS).

The thermostable chitosanase does not need to be purified in order to be used in the method. For example, a sample of a culture medium that was previously cultured with a host cell capable of expressing (and preferably secreting) a thermostable chitosanase can be used. However, the thermostable chitosanase may be purified, in part, in order to be included in the method. Purification means that may be used include, but are not limited to, bacterial lysis, centrifugation, precipitation, filtration, dialysis, solvent extraction, electrophoresis, lyophilization and/or chromatography.

Even though the optimal temperature of the thermostable chitosanase is 80° C., the method is preferably conducted at a temperature between 55° C. to 60° C. (for reactions lasting between 24 and 48 hours) or between 70° C. and 75° C. (for reactions lasting between 10 min and 6 hours). Higher temperatures facilitate the dissolution of chitosan in aqueous solutions. However, mixtures of chitosan molecules, especially concentrated mixtures of chitosans of lower molecular weight are subjected to the Maillard's reaction at high temperatures, resulting in brownish, chemically altered products which are inadequate for many applications. The occurrence of this reaction sets the upper temperature limits for enzymatic hydrolysis of chitosan at 70 to 75° C. for reaction times between 10 min and 6 h and 55 to 60° C. for reaction times between 24 hours and 48 hours.

Chitosanase Csn1794 retains at least 50% of its activity for more than 20 hours at 70° C. This property makes the enzyme suitable for the hydrolysis of chitosan at the highest temperatures recommended for a given process (to limit Mallard's reaction). Such a long life property at high temperatures in the presence of substrate has not been shown so far for any known chitosanase.

In a scaling-up processes (1 L batch), it was determined that 1 U per g of chitosan of the chitosanase Csn1794 incubated at 60° C. for 24 hours was necessary to generate chitosan oligosaccharides.

The thermostable chitosanase is able to cleave a variety of different chitosan molecules. Chitosan is a linear polysaccharide composed of randomly distributed β-(1-4)-linked D-glucosamine (deacetylated unit) and N-acetyl-D-glucosamine (acetylated unit). Typically, the chitosan molecules are defined by their length as well as their degree of deacetylation or DDA. Chitosan which are commonly used in the industry usually have a DDA of more than 50%, and usually 70% or more.

As indicated above, the thermostable chitosanase retains its enzymatic activity over a relatively large pH range, e.g. between 3.8 and 6.5. The method is preferably performed at a pH between 4.2 and 4.8, the optimal pH of the thermostable chitosanase.

The present invention will be more readily understood by referring to the following examples which are given to illustrate the invention rather than to limit its scope.

### EXAMPLE I

### Isolation and Characterization of Bacteria Producing Thermostable Chitosanase

In order to find a microorganism producing a thermostable chitosanase enzyme which could be used for large-scale production of low molecular weight chitosans (LMWC) or of chitooligosaccharides (CHOS), different types of composts were screened. Among about two thousands of microbial isolates, the isolate 1794 (also referred to as strain 1794) was selected as the strain producing the chitosanase with the highest thermostability following a multi-step screening and selection procedure involving micro-plate assays of chitosanase activity at various temperatures.

The screening was first performed on a compost material based on shrimp shells and peat, using the following procedure. Eight (8) g of compost (wet weight) were combined with 1 g of chitosan (Sigma) flakes and thoroughly mixed. The mix (compost and chitosan) was wetted with a salt-buffer solution containing Bushnell Haas salts (3.27 g/l) and K₂HPO₄ (6.44 g/l); pH 6.5-6.8. The mix was incubated at 37° C. for 6 weeks with periodic wetting. At the end of this incubation, a further selective treatment was applied to the mix. One (1) g of the mix was combined with 10 ml of a 2.5% aqueous phenol solution. After vigorous vortexing, the suspension was incubated for 30 min at 50° C. The suspension was then centrifuged (4000 rpm, 10 min) and the pellet was suspended in 10 ml of peptone water and vortexed. Various dilutions of the suspension were plated on Tryptone-Yeast Extract-Glucose agar medium pH 6.5 (3,27 g/l Bushnell Haas salts; 2.5 g/l Tryptone; 1.25 g/l Yeast Extract; O.5 g/l Glucose; 5 g/l NaCl; pH 6,5). After 48 h of incubation at 37° C., individual colonies were picked up and their ability to produce chitosanase was tested in a liquid medium with chitosan. Chitosanase activity was assayed with the soluble, dyed substrate sRBB-C (Zitouni et al., 2010). Strain 1794 was selected as having the most thermostable chitosanase activity.

Strain 1794 was maintained on tryptic soy agar plates (Difco) and routinely propagated in tryptic soy broth (Difco) at 45° C. with shaking. For long term storage, a loop of cells from the agar plate was inoculated in tryptic soy broth and grown overnight at 45° C. The culture was combined with an equal volume of sterile glycerol, then dimethyl sulfoxide was added to final concentration of 5% (v/v) and the cell suspension was kept frozen at −80° C.

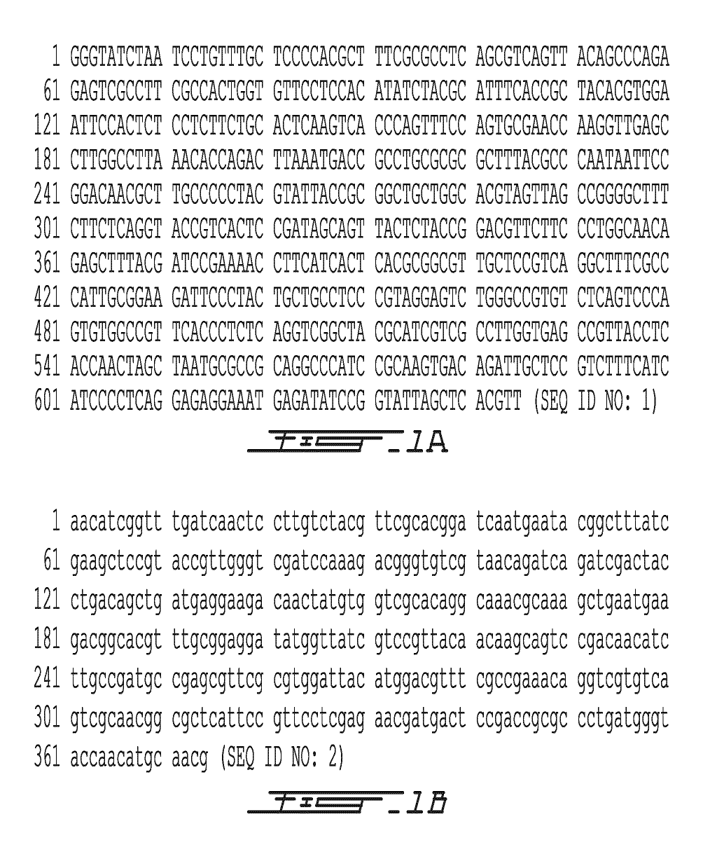

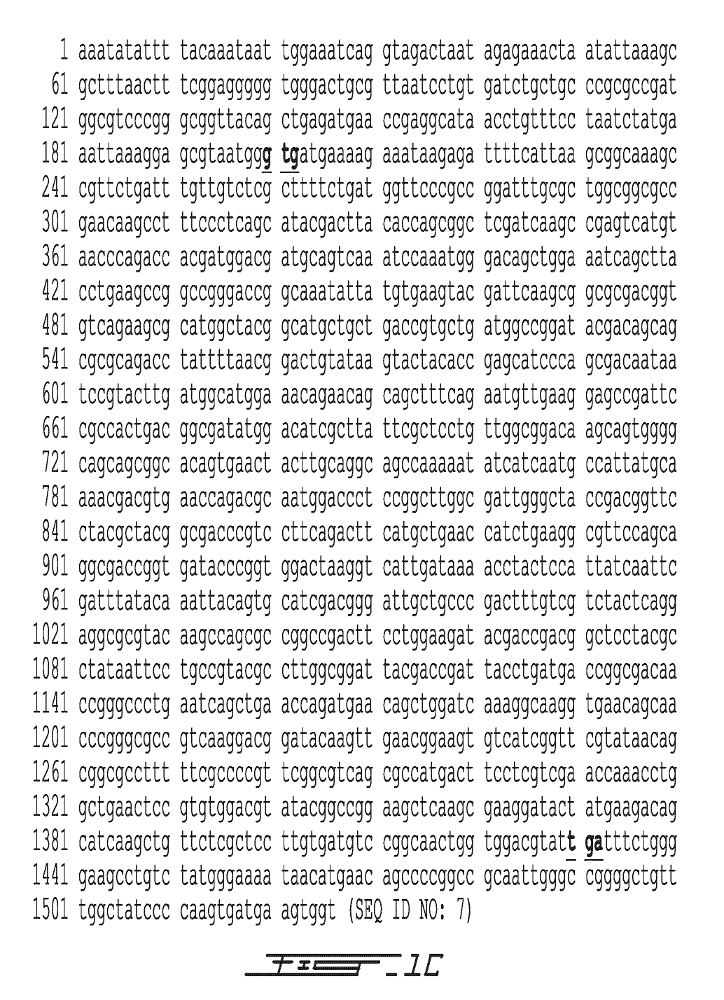

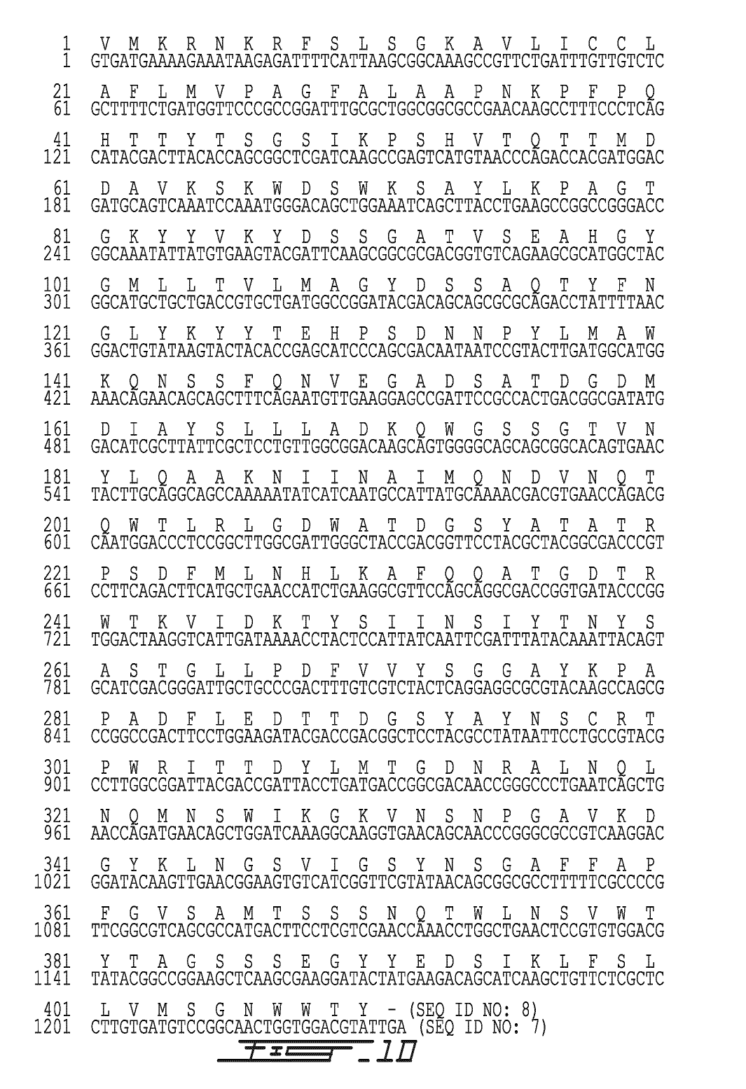

The newly isolated strain 1794 has been classified following morphological, biochemical and molecular criteria. As the first step in identification, molecular criteria were applied. The sequence of the hyper-variable portion of the gene encoding the ribosomal 16S RNA has been determined (FIG. 1, SEQ ID NO: 1). The sequence allowed concluding that the isolate 1794 is a member of the genus Paenibacillus, however, it did not allow to distinguish this isolate from other, already described paenibacilli such as P. favisporus and P. cineris. While the sequences of 16S rRNA are still widely used for bacterial identification, the recently developed methods based on partial sequencing of the rpoB gene encoding the beta subunit of the RNA polymerase offer a greater resolution, allowing distinguishing among closely related species. The so-called DGGE segment of the rpoB gene was sequenced for the 1794 isolate (FIG. 2, SEQ ID NO: 2). Comparison of this sequence with the sequences available in the GenBank database showed that the isolate 1794 represents a novel species of the genus Paenibacillus, being distinct from all known isolates. The novelty of the 1794 isolate was also confirmed by a set of selected biochemical tests. The main biochemical characteristics of strain 1794 are described in Table 1.

TABLE 1
Biochemical/morphological characteristics of strain 1794

| Characteristic | Comments |
|---|---|
| Morphology | Bacillus-like; about 1 × 0.2 μm; |
| | unicellular form not aggregated in |
| | liquid culture, short chains |
| | sometimes visible in material |
| | taken from colonies on agar plates |
| Gram coloration | Gram-positive |
| Other | Spores present in old cultures |
| Optimum growth temperature | 45° C. |
| Catalase | Positive |
| Oxidase | Positive |
| Tyrosinase | Negative |
| Caseinase | Positive |
| Amylase | Positive |
| Beta-galactosidase | Positive |
| Growth at 50° C. | Positive |
| Growth at 55° C. | Positive |
| Growth in the presence of 5% NaCl | Positive but weak |
| Methyl Red test | Negative |
| Voges-Proskauer test | Negative |
| Gas production from glucose | Negative |
| Haemolysis | Negative |
| Sugar utilization: | |
| Mannitol | Positive |
| Sorbitol | Positive |
| Acid from organic compounds: | Positive |
| D-glucosamine | Positive |
| N-acetyl-D-glucosamine | Positive |
| L-arabinose | Positive |
| Glycerol | Negative |
| Sorbitol | Negative |
| D-Ribose | Positive |
| D-xylose | Positive |
| D-glucose | Positive |

### EXAMPLE II

### Chitosanase Characterization

Chitosanase production was increased by the 1794 isolate when a medium containing oligomeric chitosan derivatives as carbon source was used. A 12 h to 18 h of Paenibacillus sp. 1794 pre-culture was prepared by inoculation of a loop of this strain into Tryptic soy broth (Difco) and incubation with shaking (300 rpm) for 14 to 18 h at 45° C. The chitosanase production medium was then prepared as follows. First, a 2.5-times concentrated base medium was obtained by dissolving in distilled water: peptone (12.5 g/L), K₂SO₄ (2.5 g/L), K₂HPO₄ (12.5 g/L), MgSO₄ (2.5 g/L). NaCl (0.25 g/L), CaCl₂ (400 mg/L), FeSO₄ (25 μM), and 0.5 ml of microelements solution (ZnSO₄*7H₂O, 1 g/L; MnCl₂*4H₂O, 1 g/L). This concentrated base medium was sterilized by autoclaving. Final pH after sterilization was around 6.5, usually without further adjustment. Separately, sucrose, chitosan oligosaccharides and D-glucosamine solutions in distilled water (each concentrated at 100 g/L) were prepared and filter sterilized. Still separately, an aqueous suspension of chitosan from Sigma (degree of deacetylation around 82%) consisting of 5 g of finely ground chitosan flakes suspended in 390 ml of distilled water, was sterilized by autoclaving and left for later use.

To reconstitute the chitosanase production medium (1 L), 400 mL of concentrated base medium were mixed with 100 mL of D-glucosamine solution, 70 ml of chitosan oligosaccharides solution and 40 mL of sucrose solution. The pH of the medium after the addition of carbohydrate solutions was still close to 6.3.

The medium reconstituted in such a way was inoculated in a 1/10 proportion with the pre-culture of the 1794 isolate and incubated at 45° C. with shaking (300 rpm). After 10 h of incubation, the culture was combined with the sterile aqueous chitosan suspension (390 mL) thus giving the final chitosanase production medium. The culture was further incubated for 5 to 7 days. At the end, the bacterial cells were eliminated by centrifugation and the supernatant kept for enzyme purification.

Chitosanase recovery by precipitation. Bacterial cells were eliminated from the culture (500 mL) by centrifugation or filtration. The culture supernatant was then cooled to 4° C. The supernatant was then combined with 2.5 volumes of ice-cold ethanol with constant mixing. The mixture was left to precipitate for 18 hours. The precipitation supernatant was then carefully removed with a pump. The pellet was suspended in 25 mL of 20 mM Tris-HCl buffer pH 8.0 and left to dissolve overnight at 4° C. The rather viscous resulting solution was extracted 3 times with 10 mL of chloroform and centrifuged to eliminate undissolved particles. The crude enzyme preparation was kept at 4° C. until used. For long term storage, the crude enzyme preparation was combined with 1 volume of sterile glycerol and kept at −20° C.

Chitosanase purification by ion exchange chromatography. The crude enzyme solution obtained after the precipitation step was diluted 1: 1 with 20 mM Tris-HCl buffer pH 8.0 and loaded on a Q-Sepharose Fast Flow™ column (25 mL of bed volume) (GE Healthcare). After washing of the column with 50 mL of loading buffer, the proteins were eluted with a 400 mL gradient of NaCl (from 0 to 1 M) in 20 mM Tris-HCl pH 8.0. Fractions of 5 ml were collected. The active fractions were identified by assay with sRBB-C substrate (as described in Example I) and pooled.

Chitosanase concentration (facultative). The pooled fractions from the previous purification step (25 mL) were diluted with four volumes of 20 mM Tris-HCl buffer pH 8.0 and loaded on a mini-column (bed volume 1 ml) Hi-Trap Q™ (GE Healthcare). Chitosanase was eluted with 0.5 M NaCl in 20 mM Tris-HCl pH 8.0. Fractions of 0.2 mL were collected. The active fractions were identified by assay with sRBB-C substrate (as described in Example I) and pooled.

Chitosanase purification by hydroxyapatite chromatography. Fractions from the concentration step (1.2 mL) were combined with ten volumes of 1 mM potassium phosphate buffer pH 6.8 and loaded on a hydroxyapatite column (BioRad; DNA grade; bed volume 10 mL) equilibrated with the same buffer. After washing of the column with 20 mL of the loading buffer and 20 mL of 5 mM unbuffered MgCl₂, the proteins were eluted with a 40 mL gradient of 1-300 mM potassium phosphate buffer pH 6.8. Fractions of 0.5 mL were collected. The active fractions were identified by assay with sRBB-C substrate (as described in Example I) and pooled. The resulting chitosanase Csn1794 preparation was homogenous, as estimated from polyacrylamide electrophoresis gel stained with Coomassie Brilliant Blue (data not shown). The purified enzyme solution was dialyzed against 50 mM sodium acetate buffer pH 4.5 and stored at 4° C. in a polycarbonate tube. As an example, a solution having an activity of 19 units per mL was stored for a two-years period without any loss of activity.

Chitosanase enzymatic activity assay. Chitosanase standard activity assay contained 480 μL of chitosan solution (0.8 mg/mL chitosan dissolved in 50 mM Na-acetate buffer pH 4.8) and 20 μL of appropriately diluted enzyme sample. The mixture was incubated for 10 min at 37° C. The reaction was terminated by the addition of 1.0 mL of Lever reagent (1% p-hydroxybenzoic acid hydrazide in 1.25 M NaOH). After 20 min of incubation in a mineral oil bath at 100° C., chilling in ice water and centrifugation (in order to eliminate the chitosan precipitate), soluble reducing sugars were determined spectrophotometrically at 405 nm using a standard curve of D-glucosamine. One unit of enzyme was defined as the amount that liberated 1 μmol of D-glucosamine equivalent in 1 minute under the above conditions.

Csn1794 substrate specificity. The purified Csn1794 enzyme hydrolyzed chitosans of various degrees of deacetylation, carboxymethyl cellulose and methylcellulose. No activity was detected against microcrystalline cellulose, chitin, xylan, laminarin, pustulan, pachyman, mannan, or β-1,3-1,4-glucan. If Csn1794's activity against chitosan is considered as a 100% reference value, Csn1794's activity against carboxymethyl cellulose is estimated at 58% and against methylcellulose at 14%.

Starting with a highly deacetylated chitosan (DDA=99%; obtained from Marinard, Rivière-au-Renard, Québec, Canada), a series of chitosan having lower degree of deacetylation (DDA) values were prepared by a treatment with acetic anhydride according to a published procedure (Côté et al, 2006). The activity of Csn1794 was then assessed for these chitosan preparations. In contrast with many known chitosanases, the chitosanase activity of Csn1794 varies little with the degree of deacetylation of its substrate (FIG. 2). The chitosanase Csn1794 is thus able to hydrolyze efficiently a wide spectrum of chitosan substrates.

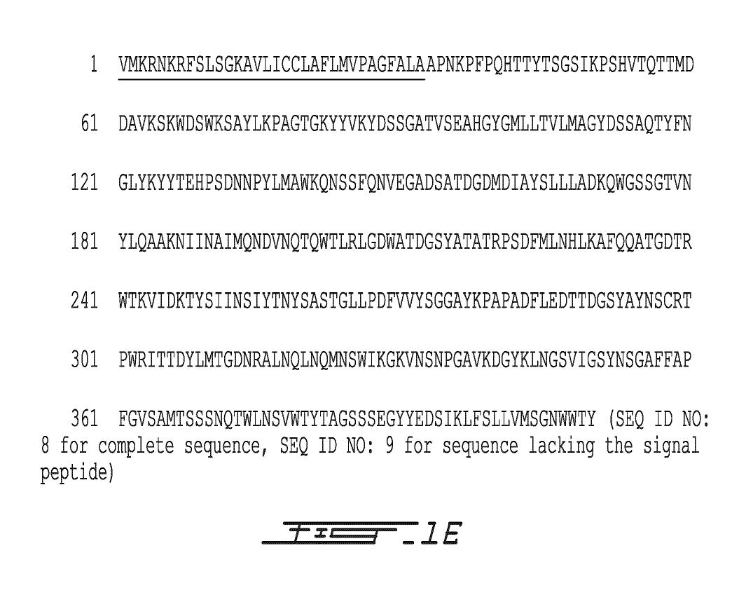

The activity of Csn1794 was assayed with a series of buffers in the mildly acidic range. The purified enzyme has been combined with chitosan Sigma (DDA=84%) buffered with Na-acetate at various pH. Reaction was incubated for 10 min. at 37° C. Optimal pH is around 4.7-4.8, but the enzyme kept at least 80% of activity in a wide range of pH values between 3.8 and 6.5 (FIG. 3).

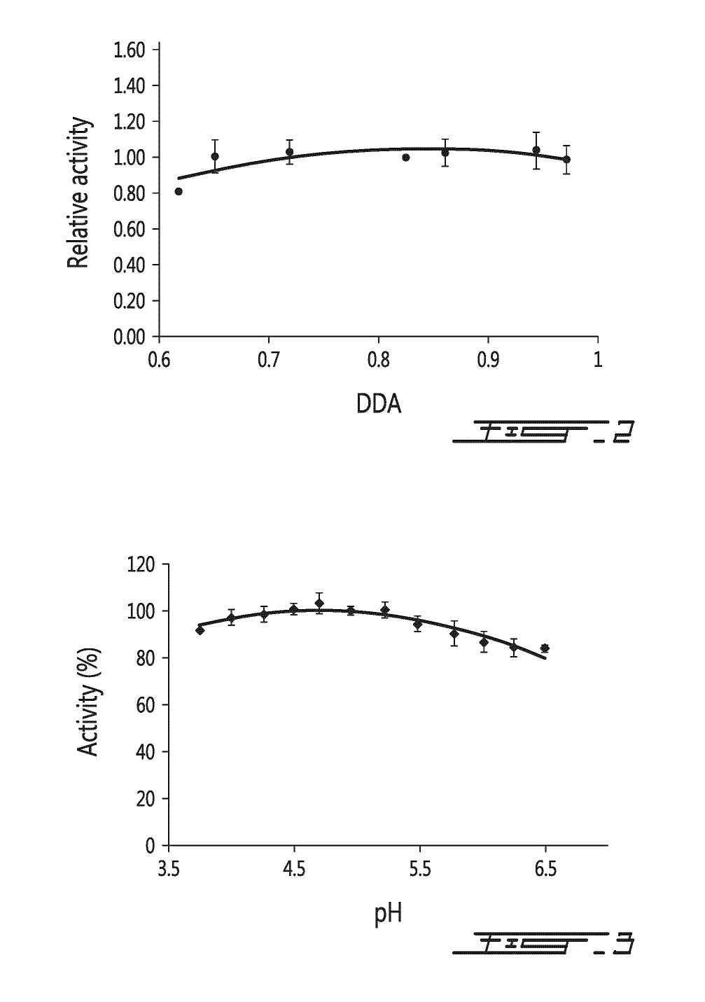

The effect of temperature on enzyme activity was determined by running assays at 3, 5 or 10 min. Maximal activity was observed at 80° C. for 10 min and 85° C. for 5 min and 3 min incubation times (FIG. 4). These values are among the highest observed for known chitosanases.

The concentration of the purified protein can be determined spectrophotometrically, measuring the absorption at 280 nanometers and using the value of molar absorption of 114701 (established from the amino acid composition of this protein) for calculation.

On this basis, the specific activity of Csn1794 at 37° C. was found to be equal to 122.4 units per mg of protein.

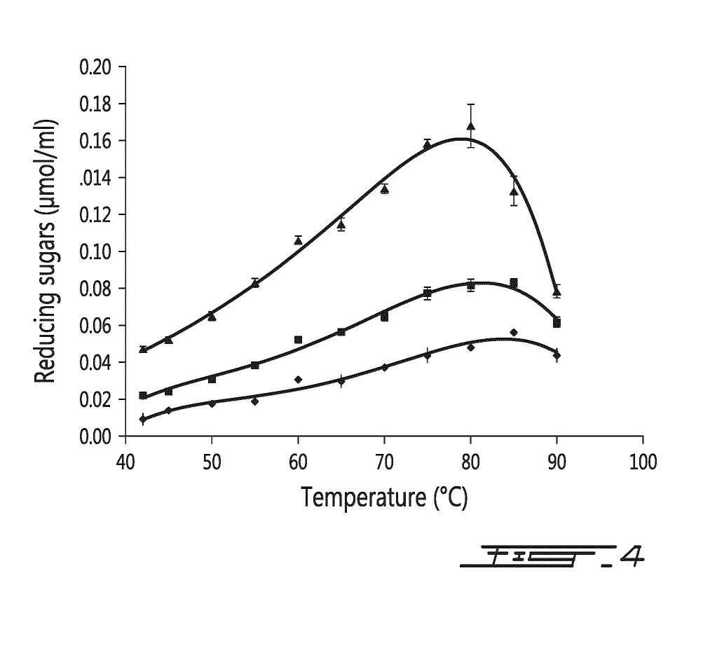

The apparent Kₘ and the catalytic constant k_{cat} were calculated using a weighted least-squares fit approach. The behaviour of Csn1794 chitosanase was well represented by the model assuming that substrate inhibition influenced the rate of chitosanase-catalyzed reaction (FIG. 5) yielding the following values: Kₘ=0.042 mg/ml; k_{cat}=7588 min⁻¹; Kᵢ=1.66 mg/ml.

As several chitosanases were shown to be stabilized by the presence of chitosan substrate, the deactivation kinetics of chitosanase Csn1794 has been estimated directly in the conditions of a chitosan hydrolysis bioreactor. Assuming a first-order deactivation kinetics, the half-life of chitosanase at a given temperature can be estimated from the time-course of product accumulation. The enzyme half-life is defined as the period of time required to inactivate the enzyme at 50% of initial activity.

For the purpose of half-life estimation, chitosan substrate (Sigma) has been dissolved at a concentration of 5 g/L in acetic acid, which concentration has been adjusted to obtain a final pH of 4.4-4.6. Temperature was set up at 70, 75 or 78° C. After the addition of chitosanase (8 milliUnits per mL), samples were taken periodically for a time up to 6 hours and the accumulation of product was determined by the reducing sugars assay with Lever reagent. The time-course curve was analyzed by non-linear regression software, in order to determine the best fitted value of the deactivation constant (K_{d}), and chitosanase half-life (T_{1/2}) was calculated using the following equation:

T_{1/2}=0.693/K_{d}

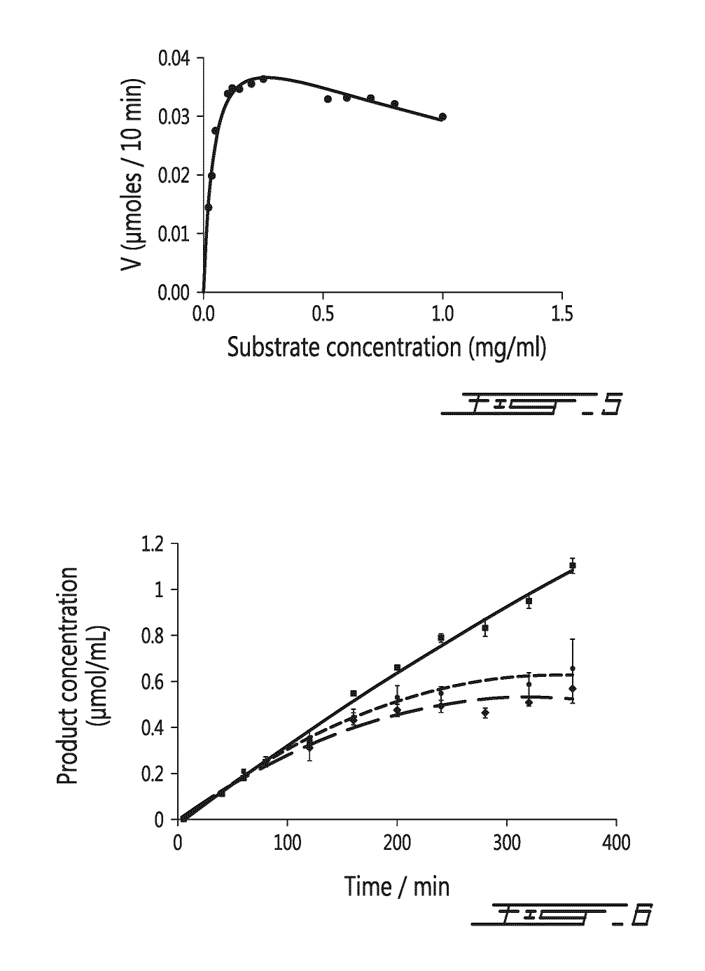

The following chitosanase half-life values were found by analysis of time-course curves shown in FIG. 6: 1387 min at 70° C.; 129 min at 75° C.; 108 min at 78° C.

### EXAMPLE III

### Sequence Determination of Csn1794

Chromosomal DNA was extracted from Paenibacillus sp. 1794 using a method involving lysozyme-aided cell lysis. RNAse and Proteinase K treatment.

A DNA probe suitable for the identification of the DNA fragment carrying the csn1794 gene was identified by a reverse genetics approach as follows. The purified Csn1794 protein was digested into short peptides with trypsin and the sequence of several peptides has been determined. Based on the sequence of two peptides (N terminus-K W N S W K-C terminus (SEQ ID NO: 3) and N terminus-T T D Y L M-C terminus (SEQ ID NO: 4)), two synthetic oligonucleotide primers have been designed according to the principle of reverse translation:

(SEQ ID NO: 5)
Primer 1: 5′-AAATGGAACAGCTGGAAA-3′
(SEQ ID NO: 6)
Primer 2: 5′-CATCAGATAGTCGGTCGT-3′

A polymerase chain reaction has been set up with these two primers, allowing to amplify a 648-base pairs DNA fragment, representing an internal segment of the csn1794 gene coding sequence. The amplified DNA fragment was labeled with digoxigenin and used as a hybridization probe with genomic DNA fragments of Paenibacillus sp. 1794 obtained by treatment with BspEl restriction enzyme (New England Biolabs Inc.) and separated by agarose gel electrophoresis.

The hybridization showed that the csn1794 gene is localized on a BspEl fragment of an approximate length of 6 kilobase pairs. A partial genomic bank has been constructed by extraction of BspEl-digested genomic fragments of the appropriate size from the agarose electrophoresis gel and ligation into the Litmus 38i plasmid pre-digested with BspEl restriction enzyme. After ligation, the plasmids were transformed into an appropriate Escherichia coli host (E. coli DH5α available from American Type Culture Collection (ATCC 47093)). The transformants were plated on petri plates containing bacto-tryptone (10 g/L), yeast extract (5 g/L), NaCl (5 g/L), agar (15 g/L) and ampicillin (100 mg/L) and incubated for 24 h at 37° C.

After incubation, the colonies were collected from the surface of the plates and bacteria were suspended in sterile solution of 0.9% w/v NaCl. After appropriate dilution, 100 μL portions of the suspension were plated on chitosanase detection medium (Zitouni et al., 2010) prepared as follows. A basal medium was prepared containing MgSO₄ (0.2 g/L), CaCl₂ (0.02 g/L), K₂HPO₄ (1 g/L), KH₂PO₄ (1 g/L), FeCl₃ (0.05 g/L), NaCl (2.5 g/L), glucose (1 g/L), (NH₄)₂SO₄ (1 g/L), malt extract (Difco) (1 g/L), NaOH (5 mM) and agar (15 g/L), pH 6.5. 900 mL of water were added and the medium was autoclaved. Separately, the soluble Remazol Brilliant Blue™ chitosan (Zitouni et al., 2010) was dissolved at a concentration of 5 g/L in 100 mL of 50 mM HCl and autoclaved. After sterilization, the hot agar medium (60° C.) was combined with the sRBB-C solution and poured on petri plates. The inoculated plates were incubated for 72-96 h at 37° C.

Chitosanase-positive colonies were identified by the formation of a clear halo around the colony.

A plasmid clone contained in a positive colony has been sequenced. The nucleotide sequence of the 5.8 kilobase pairs insert carried by the chitosanase-positive Escherichia coli clone has been determined, and shown to carry the complete csn1794 gene (FIG. 1C and SEQ ID NO: 7). FIG. 10 shows the chitosanase-encoding DNA sequence together with upstream and downstream intergenic segments. The open-reading frame (ORF) encoding the chitosanase starts at nucleotide 200 and ends at nucleotide 1432. This ORF determines a protein of 410 amino acids, corresponding to the intracellular precursor of chitosanase. FIG. 1D shows the chitosanase-encoding DNA sequence (SEQ ID NO: 7) together with its translation into amino acids (SEQ ID NO: 8). FIG. 1E shows the amino acid sequence of the chitosanase Csn1794 protein with a peptide signal (underlined, SEQ ID NO: 8) and without a peptide signal (SEQ ID NO: 9). As the chitosanase Csn1794 is an extracellular protein, the signal peptide localized at the N-terminus is cleaved out during the secretion process. Using a bioinformatic procedure (Emanuelsson et al., 2007), the signal peptidase cleavage site is predicted to be localized between residues 32 and 33 of the precursor protein, resulting in a secreted, mature form of chitosanase beginning with residue Ala-33 at its N-terminus (SEQ ID NO: 9).

Accordingly, the extracellular, mature form of active chitosanase is a protein composed of 378 amino acids with a calculated molecular weight of 41.8 kilodaltons. The calculated molecular weight corresponds to that measured by SDS-polyacrylamide gel electrophoresis.

### EXAMPLE IV

### Production of Low Molecular Weight Chitosan (LMWC) and Chitosan Oligosaccharides

Low Molecular Weight Chitosan Production. Native, powdered chitosan (60 g—Marinard inc., Rivière-au-Renard; Québec, Canada) is suspended in 900 mL of water and left for a few minutes with moderate mixing. Then, glacial acetic acid is added to a final concentration of 0.4 M and the total volume is completed to 1000 mL with water. The solution is left overnight at room temperature with mixing. The pH of the resulting solution is around 4.5. The viscous solution is warmed up to 65° C. and 5 units of chitosanase are added. The number average molecular weight of the hydrolyzed chitosan (Mn) is estimated by the reducing sugars assay using the Lever reagent.

Chitosan of Mn of 45 kilodaltons is obtained after 40 minutes of hydrolysis. Chitosan of Mn of 26 kilodaltons is obtained after 120 minutes of hydrolysis. The progressive reduction of molecular weight as well as the very low level of monomeric D-glucosamine resulting from hydrolysis (as measured by a modified Morgan-Elson assay) indicate that the chitosanase Csn1794 is an endo-chitosanase, breaking internal bonds in the chitosan chain.

Chitosan Oligosaccharides Production. Native, powdered chitosan (50 g—Marinard inc., Rivière-au-Renard; Québec, Canada) is suspended in 900 mL of water and left for 2 hours with moderate mixing. Then, glacial acetic acid is added to a final concentration of 0.4M and the total volume is completed to 1000 mL. The solution is left overnight at room temperature with mixing. The pH of the resulting solution is around 4.5. The viscous solution is warmed up to 60° C. and 50 units of chitosanase are added. The average degree of polymerization (DP) is calculated from the number average molecular weight of the hydrolyzed chitosan (Mn), estimated by the reducing sugars assay using the Lever reagent. The decrease of the degree of polymerization of the hydrolyzed chitosan during hydrolysis is shown in Table 2.

After 24 h of hydrolysis, the mixture is essentially composed of dimeric and trimeric chitooligosaccharides. This composition is close to the end product expected for chitosanases with endo-mechanism. A low proportion (0.5-1%) of monomeric D-glucosamine was present in the hydrolyzate, as measured by a modified Morgan-Elson assay (Côté et al., 2006).

TABLE 2
Degree of polymerization of hydrolyzed chitosan obtained
after various times of hydrolysis at 60° C. Chitosanase
was added in the ratio of 1 unit per gram of chitosan.

| | Degree of |
|---|---|
| Time of hydrolysis | polymerization |
| 1 h | 23 |
| 2 h | 14 |
| 4 h | 7 |
| 6 h | 4.5 |
| 12 h | 3.1 |
| 24 h | 2.7 |
| 30 h | 2.6 |

### REFERENCES

Côté, N., Fleury, A., Dumont-Blanchette, E., Fukamizo, T., Mitsutomi, M., Brzezinski, R. (2006) Two exo-β-D-glucosaminidases from actinomycetes define a new subclass of family 2 of glycoside hydrolases. Biochemical Journal 394:675-686.
Emanuelsson, O., Brunak, S., von Heijne, G., Nielsen, H. (2007) Locating proteins in the cell using TargetP, SignalP, and related tools. Nature Protocols 2: 953-971.
Roberts, G. A. F (1992) Chitin chemistry. The MacMillan Press, London,UK; ISBN 0-333-52417-9.
Zitouni, M., Fortin, M., Thibeault, J.-S., Brzezinski, R. (2010) A dye-labelled soluble substrate for the assay of endo-chitosanase activity. Carbohydrate Polymers 80:522-525.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An isolated thermostable chitosanase comprising an amino acid sequence having more than 82% identity to the amino acid sequence of SEQ ID NO:9, wherein said chitosanase has an optimal temperature of about 80° C. when measured at a reaction time of about 10 minutes.

2. The isolated thermostable chitosanase of claim 1, further comprising at least one of a glutamate residue corresponding to position 64 or a glutamate residue corresponding to position 254.

3. The isolated thermostable chitosanase of claim 1, further comprising a glutamate residue corresponding to position 64 and a glutamate residue corresponding to position 254.

4. The isolated thermostable chitosanase of claim 1, further comprising at least one of an aspartate residue corresponding to position 56, a tryptophan residue at position 108, an aspartate residue corresponding to position 121, an aspartate residue corresponding to position 125, a tryptophan residue corresponding to position 177, a tyrosine residue corresponding to position 263 or a tyrosine residue corresponding to position 359.

5. The isolated thermostable chitosanase of claim 1, further comprising an aspartate residue corresponding to position 56, a tryptophan residue corresponding to position 108, an aspartate residue corresponding to position 121, an aspartate residue corresponding to position 125, a tryptophan residue corresponding to position 177, a tyrosine residue corresponding to position 263 and a tyrosine residue corresponding to position 359.

6. An isolated thermostable chitosanase comprising an amino acid sequence having more than 81% identity to the amino acid sequence of SEQ ID NO:9, wherein said chitosanase has an optimal temperature of about 80° C. when measured at a reaction time of about 10 minutes, wherein the amino acid sequence of the isolated thermostable chitosanase comprises a glutamate residue corresponding to position 64, a glutamate residue corresponding to position 254, an aspartate residue corresponding to position 56, a tryptophan residue corresponding to position 108, an aspartate residue corresponding to position 121, an aspartate residue corresponding to position 125, a tryptophan residue corresponding to position 177, a tyrosine residue corresponding to position 263 and a tyrosine residue corresponding to position 359.

7. The isolated thermostable chitosanase of claim 1 being derived from Paenibacillus sp. 1794 deposited at the International Depository Authority of Canada (IDAC) on January 11, 2011 and having IDAC Accession Number 110111-01.

8. An isolated nucleic acid molecule encoding the isolated chitosanase of claim 1.

9. The isolated nucleic acid molecule of claim 8, comprising nucleotides 200 to 1432 of the nucleotide sequence of SEQ ID NO:7.

10. (canceled)

11. A vector encoding the isolated chitosanase of claim 1.

12. (canceled)

13. A host cell expressing a recombinant form of the isolated thermostable chitosanase of claim 1.

14. The host cell of claim 13, further comprising an isolated nucleic acid molecule encoding the isolated chitosanase.

15-18. (canceled)

19. A method of reducing the molecular weight of a chitosan molecule, said method comprising contacting the isolated chitosanase of claim 1 with said chitosan molecule under conditions sufficient to allow the cleavage of said chitosan molecule by said isolated chitosanase.

20. The method of claim 19, wherein the contacting occurs at a temperature of between about 60° C. to about 80° C.

21. The method of claim 19, wherein the contacting occurs at a temperature of about 70° C.

22-29. (canceled)
